(19) United States
(12) Patent Application Publication
(10) Pub. No.: US 2002/0015965 A1
(43) Pub. Date: Feb. 7, 2002

(54) Efficient synthesis of triboluminescent lanthanide complexes

(76) Inventor: Linda Marie Sweeting, Baltimore, MD (US)

Correspondence Address: Royal W. Craig, Law Offices of Royal W. Craig, Suite 1319, 210 N. Charles Street, Baltimore, MD, 21201, US

(21) Appl. No.: 09/917,308

(22) Filed: Jul. 27, 2001

Related U.S. Application Data
(60) Provisional application No. 60/220,992, filed on Jul. 27, 2000.

(51) Int. Cl. G01N 33/53; C07F 5/00

(52) U.S. Cl. 435/7.1; 534/15

(57) ABSTRACT

Lanthanide complexes of β-diketonates which incorporate amines or their salts have been shown to exhibit intense photoluminescence and triboluminescence. These triboluminescent materials show a great deal of promise for remote sensing of strain, impact, pressure and fracture. The present invention is a new and more efficient process for synthesizing and testing triboluminescent and photoluminescent materials.

### CROSS-REFERENCE TO RELATED APPLICATIONS

The present application derives priority from U.S. Provisional Patent Application 60/220,992 for “Methods for Synthesizing Triboluminescent Lanthanide Complexes and New Triboluminescent Lanthanide Complexes Discovered Thereby”; Filed: Jul. 27, 2000; Applicant: Linda M. Sweeting.

### BACKGROUND OF THE INVENTION

1. Field of the Invention

This invention relates to triboluminescent materials and, more particularly, to lanthanide complexes of β-diketonates which incorporate amines or their salts and which have been shown to exhibit intense photoluminescence and triboluminescence.

2. Description of the Background

Triboluminescence is a well documented phenomenon wherein light is emitted from certain materials, often crystalline compounds, subjected to mechanical stress [See Walton, A. J. Advances in Physics 1977, Vol. 26, No. 6,887-948]. Triboluminescent materials have a great deal of potential for examination of the mechanisms and processes of deformation and failure of materials which are not themselves triboluminescent. Added chemiluminescent materials have been used to study polymer mechanical processes [Fanter, D. L.; Levy, R. L. In Durability of Macromolecular Materials; ACS Symposium Series 95; American Chemical Society: Washington, D.C., 1979]. Even polymers which are triboluminescent may benefit from the enhancement of the signal or monitoring the effect of impurities or the selection of failure modes by the energy needed using an added triboluminescent sensor molecule.

Triboluminescent materials have a great deal of potential for use in a variety of applications as optical sensors of fracture, stress, impact or pressure. Triboluminescent materials have been incorporated into prototype devices for monitoring flexure and impact, with color coding of location by selection of the lanthanide and fiber-optic transmission to a monitoring station [Sage, I.; Badcock, R.; Humberstone, L.; Geddes, N.; Kemp, M.; Bourhill, G. Smart Mater. Struct. 1999, 8,504-510]. Others have suggested using them as coatings to detect mechanical stress [Xu, C. N.; Watanabe, T.; Akiyama, M.; Zheng, X. G. Appl. Phys. Lett. 1999, 74,1236-1238] or embedded to detect loading [Xu, C. N.; Watanabe, T.; Akiyama, M.; Zheng, X. G. App/.Phys. Lett. 1999, 74,2414-2416] with the emitted light pattern visualizing the i pattern of stress [Xu, C. -N.; Zheng, X. -G.; Akiyama, M.; Nonaka, K.; Watanabe, T. Appl. Phys. Lett. 2000, 76,179-181].

Devices currently using alternate technologies are adaptable to triboluminescent materials for remote sensing. Embedded fibers are used for detection of wear without the triboluminescence, using visual observation of the fiber exposure. Triboluminescence would improve this device significantly both by calling attention to the worn area, and by indicating the degree of wear as measured by the intensity of the triboluminescence. The device could be used on automobile tires, and thus provide a high-tech system for identifying the need for new tires. Even more important are similar applications inside engines of all kinds, including detecting excessive wear on internal gears, shafts, and pistons in items from power plants to automobiles to home appliances.

Triboluminescence has even been suggested as a way to monitor earthquakes. Earthquake-prone zones could readily be monitored by underground sensors which emit light when strained beyond a certain predetermined limit, with the light being carried to a monitoring station.

Traditionally, the synthesis of salts of the lanthanide metals (Ln=La, Ce, Pr, Nd, Pm, Sm, Eu, Gd, Tb, Dy, Ho, Er, Tm, Yb, Lu) which are triboluminescent has been both by accident and by analogic design. In each case, traditional synthetic methods were used in which each combination of metal and organic ligands was synthesized, purified, and sequentially tested.

Hurt et al. [Hurt, C. R.; McAvoy, N.; Bjorklund, S.; Filipescu, N. Nature 1966, 212,179 180] discovered that materials made individually with Eu, Tb, and Gd according to Equation 1 (shown in FIG. 1) with a diketone in which R1=R2=phenyl were triboluminescent at room temperature with R3=phenyl and R4=R5=methyl or with R3=R4=R5=ethyl. Those wit another 14 different amines were tribolumineseent only at 77K or not at all. They found no triboluminescence if R1=trifluoromethyl and R2=2-thienyl, R1=trifluoromethyl or methyl and R2=phenyl, and others which they did not report.

Zhu et al. [Zhu, W.; Hua, W. Zhongguo Xitu Xuebao 1990, 8,102-105] discovered that materials made individually with Eu according to equation 2 (shown in FIG. 2) with a diketone in which R1=trifluoromethyl and R2=2-thienyl were triboluminescent when the amines were pyridine-N-oxide, 2-picoline-N-oxide and 2,2′-bipyridinedi-N-oxide.

Chen et al. [Chen, X. -F.; Liu, S. -H.; Duan, C. Y.; Xu, Y. -H.; You, X. -Z.; Ma, J.; Min, N. B. Polyhedron 1998, 17,1883-1889; Chen, X. F.; Zhu, X. -H.; Fun, H. -K.; Ma, J.; You, X. -Z. Inorg. Chim. Acta. 1999, 15,252-254] made triboluminescent Eu compounds according to equation 1 with a diketone in which R1=trifluoromethyl and R2=2-thienyl using the nitrate salt as the 1,4-dimethylpyridinium and N-methylisoquinolinium amine salts. You et al. also made Eu compounds with three diketones (R1=trifluoromethyl and R2=2-thienyl) and one diamine (diazafluorene, 4,4-dimethyl-2,2-bipyridine) to give a neutral salt as in Equation 2.

Takada et al. [Tanaka, N.; Sugiyama, J. -I.; Katoh, R.; Minami, N.; Hieda, S. Synth Metals 1997, 91,351-354] made triboluminescent compounds according to equation 2, using the diamine phenanthroline, unsubstituted, and substituted with 5-methyl, 5-phenyl and 5,8-diphenyl.

U.S. Pat. No. 6,071,632 to Hall-Goulle claims compounds made with Eu, Tb, Dy and Sm according to equation 2 with a diketone in which R1 and R2 are independently phenyl, and t-butyl. Hall-Goulle '632 also claims hydrogen, C₁-C₆ alkyl and an additional alkyl substituent on the center carbon of the diketonate. The compounds are triboluminescent when the amines are p-N,N-dimethylaminopyridine, N-methylimidazole and p-methoxypyridine-N-oxide and R1=R2 =C(CH3)3 for Eu, Tb, Dy, and Sm. Hall-Goulle '632 uses literature methods for making the initial lanthanide reagent. Unfortunately, the method of synthesis shown in Hall-Goulle '632, according to equation 2 (FIG. 2) requires heating the individual samples of reagents for approximately two and one-half hours in boiling ethanol to synthesize the desired compounds.

Combinatorial chemistry synthesis has emerged as an effective process for systematically synthesizing a very large number of different chemical compounds to obtain a desired product. The protocol requires an array of reaction vessels arranged according to a system comprising one or more variable parameters (e.g concentration or identity of one reagent.) Reagents are combined in the reaction vessels, and the products are screened for desired characteristics. The method has been shown to dramatically improve the efficiency of chemical synthesis and screening techniques.

The process of combinatorial chemistry, by accelerating the process of chemical synthesis, has been applied to developing new materials for catalysts, column supports, electronics, and other applications. Complex apparati for performing combinatorial synthesis protocols are known in the prior art. For example, U.S. Pat. No. 6,045,755 to Lebl et al. shows a robot apparatus having interchangeable work stations, robot arm tools, reaction vessels, and reaction vessel arrays. U.S. Pat. No. 5,980,839 to Bier et al. discloses a combinatorial vessel comprising a reaction region and a mixing region, adjacent to each other, which can be used to practice combinatorial chemical synthesis.

Combinatorial synthesis protocols have likewise been taught for the automated production of trial compounds for use as new drugs, new DNA, or protein sequences. For example, U.S. Pat. No. 6,025,371 to Gordeev et al. shows combinatorial libraries containing fused 2,4-pyrimidinediones. Gordeev '371 further provides methods for the identification of bioactive, fused 2,4-pyrimidinediones from those libraries.

### SUMMARY OF THE INVENTION

Current methods of synthesizing potentially triboluminescent materials are very time-consuming. Each compound is made individually by weighing, dissolving and mixing the reagents—a lanthanide salt, an amine and a diketone (if the latter has not been included in the initial salt). After crystals are grown, they must be filtered, rinsed, and dried, a process which takes 1-2 days per sample. Significant time is invested in each reaction procedure without any knowledge of whether the final product will even be triboluminescent. Lacking in the prior art is a method for efficiently synthesizing and screening potential triboluminscent compounds.

Accordingly, it would be useful to have an efficient and inexpensive process to synthesize and screen various diketones and lanthanides for triboluminescence. It would further be useful to provide a means to quickly identify which compounds synthesized by the foregoing process exhibit triboluminescence. The synthetic method of the present invention is especially useful because of its simplicity and speed compared to prior art. Using a combinatorial methodology, a chemical reaction having a desired molar ratio of reagents is performed at room temperature. The time and energy costs to synthesize the compounds are substantially reduced because heat is not required for the basic synthesis, no transfers are necessary, and dozens of reactions can be performed simultaneously. The combinatorial method of the present invention further improves efficiency by expediting the process for screening the product compounds for triboluminescence. The method requires no complex apparatus, and can be accomplished with basic chemical glassware.

### DESCRIPTION OF THE DRAWINGS

FIG. 1 illustrates a synthetic route to a triboluminscent amine salt product from a diketone, amine and lanthanide salt combined in a 4:4:1 molar ratio.

FIG. 2 shows the synthetic route to a triboluminscent amine salt from a lanthanide salt and amine reagent heated to reflux.

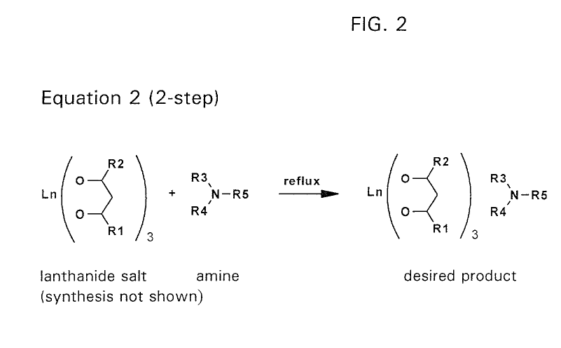

FIG. 3 shows a synthetic route to a triboluminescent amine salt from a diketone, amine, and lanthanide salt combined in a 3:4:1 molar ratio.

FIG. 4 illustrates an exemplary array of reaction vessels and starting reagents according to the combinatorial method of the present invention.

### DETAILED DESCRIPTION OF THE PREFERRED EMBODIMENTS

In one of its aspects, the present invention relates to a new room-temperature method for making triboluminescent compounds with a 3:4:1 and 4:4:1 molar ratio of reagents. The method of the present invention efficiently produces triboluminescent compounds without the step of heating the reagents. The method further includes a combinatorial methodology for synthesizing and screening the product compounds for triboluminescence without transferring them to other containers.

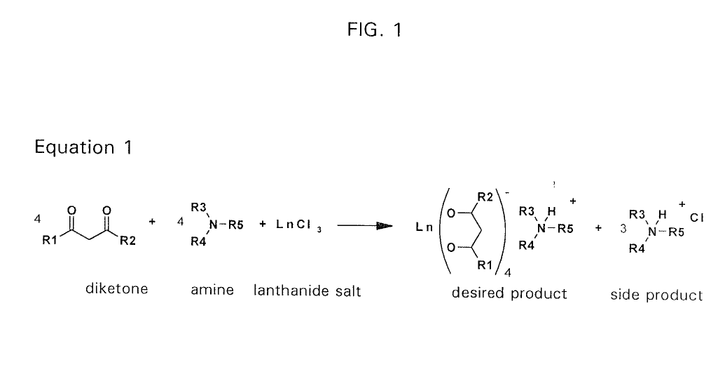

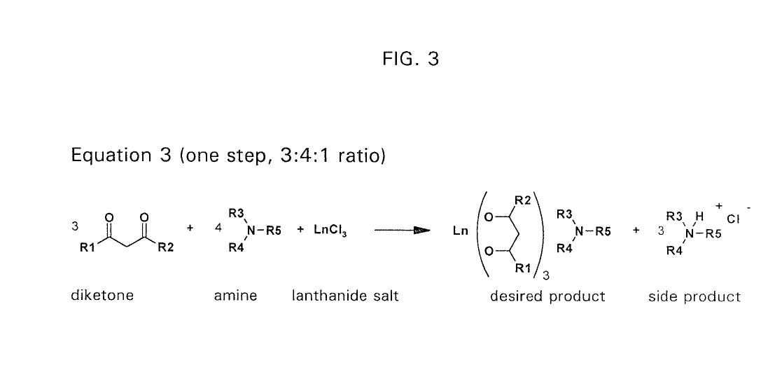

Synthesis of the triboluminescent compounds according to the present invention is described in Equation 3 (shown in FIG. 3). The reagents comprising a diketone, amine, and lanthanide salt are mixed in a 3:4:1 molar ratio at room temperature. The reaction yields the desired lanthanide complex as well as an amine salt side product which remains sufficiently soluble in the organic solvent and can be removed by rinsing or recrystalization of the desired product in the original container. The combinatorial method of the present invention is also applied to the synthesis of 4:4:1 complexes according to Equation 1 (shown in FIG. 1).

Combinatorial Synthesis

The combinatorial methodology of the present invention provides an efficient mechanism for screening the products of the test reactions for triboluminescence. It is difficult to determine a priori which combination of reagents at which concentrations will yield triboluminescent end products. The combinatorial method of the present invention enables a researcher to efficiently screen large numbers of sample reactions by systematically varying the test reagents and their concentrations over a large array of reaction vessels. Multiple products are generated and tested for triboluminescence.

The combinatorial synthesis of the present invention is a set of syntheses laid out in an array. A large number of reaction vessels, preferably test tubes, are arranged in an orderly pattern, for example in columns and rows. Test reagents are added to the individual vessels according to a pre-determined protocol. According to the protocol, a single parameter is systematically varied across the array. The parameters may include the identity of the test reagents themselves or other factors such as concentration. The result is an array of reaction mixtures, and subsequent reaction products. The method generates an organized grid of reaction products which differ according to the systematically changing variable of the experimental protocol. A researcher may thereby efficiently assess whether a reaction product is triboluminescent simply by viewing which of the reaction vessels is glowing in the array after the products are impact-tested.

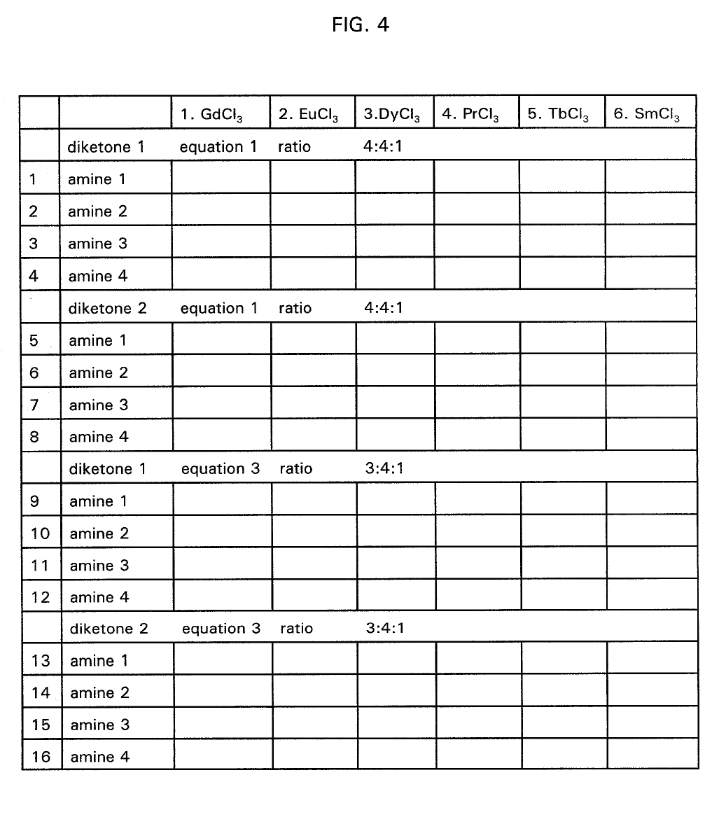

FIG. 4 is a table which illustrates an exemplary array of reaction vessels arranged according to the combinatorial methodology of the present invention. The first array shows a reaction sequence in which 24 reaction vessels are arranged in a grid comprising four horizontal rows and six vertical columns. Of course, the number and arrangement of reaction vessels may vary, and in many experiments may be much greater than the number of reaction vessels shown in the exemplary arrangement of FIG. 4. The diketone remains constant throughout the reaction sequence, and the molar ratio of 4:4:1 according to equation 1 also remains constant. Across each row, the amine remains constant, and the variable parameter is the lanthanide salt. Down each column, the lanthanide remains constant, and the variable parameter is the amine. The reaction if sequence comprises four different amines and six different lanthanide salts. Subsequent reaction sequences are performed according to the method of the present invention by varying the diketone or molar ratio of reagents (e.g. a 3:4:1: molar ratio) as shown in the arrays of FIG. 4.

Solvent is removed from the precipitates (after concentration or centrifugation, if necessary), and the precipitates allowed to dry (in air or with the assistance of heat and/or vacuum). The solids are examined by UV irradiation to determine whether the lanthanide salt formed is photoluminescent. Mechanical manipulations including crushing, fracturing, or grinding the reaction products are used to determine if the salt is triboluminescent. The method of the present invention provides for the testing of photoluminescence and triboluminescence in the original reaction vessels, without having to transfer the reaction product. The method provides a dramatic display of the reagents and conditions which successfully yield triboluminescence in each of the distinct reaction products, based on the position of each of the reaction vessels in the array. The array thereby provides a visual display of the relative triboluminescence of each of said reaction products.

The inventors showed the method to be effective by its replication of the synthesis of previously identified triboluminescent compounds which, although impure, had triboluminescence activities comparable to those made by the published method as shown by Example 2. However, because impurities can have a significant effect on triboluminescent activity, the activities of purified materials can be different. Trial recrystalizations are also accomplished in the containers in which they were made.

The following examples will facilitate a more complete understanding of the invention.

### EXAMPLE 1

Stock solutions of convenient simple lanthanide salts such as chlorides, carbonates, acetates, nitrates, fluorides dissolved in an organic solvent are prepared. Anhydrous terbium chloride; for example, is quite soluble in dry ethanol.

Stock solutions of known concentrations of the selected diketone, where R1 and R2 are independently aromatic or aliphatic are prepared. The inventors have found that bulky R1 and R2 are more likely to form solids which are triboluminescent.

Stock solutions of the selected amine, diamine or amine oxide are prepared. The present invention applies to all amines, diamines and amine oxides. Tertiary amines or pyridine-like compounds are more likely to form solids which are triboluminescent than are secondary or primary; N-oxides are also effective.

An array of sample containers, test tubes or spot plates, for example, is prepared. FIG. 4 illustrates an exemplary array.

Aliquots of the reagent solutions are added one at a time, preferably using an automatic pipette, to achieve the molar ratios of equation 1 and/or equation 3. Preferably, diketones, then amines are added before lanthanides. Other addition orders are included within the scope of the invention.

Most of the supernatant liquid is removed from any solid that forms by pipetting or decanting, with or without prior centrifugation. Rinsing the solid with an additional aliquot of solvent and removing same before complete drying provides a more reproducibly characterizable product, but is not necessary. The solid is dried in air, with or without the aid of vacuum or heat.

The photoluminescence of the crude material is tested with an UV lamp or suitable spectrometer. The triboluminescence of the crude material is tested by inserting a rod or spatula into the reaction container and impacting or grinding the reaction product. Alternatively, mechanical devices may be used.

To improve the purity for full characterization and practical application, samples are recrystalized in the test tube to see if triboluminescence activity changes. For those samples which still exhibit triboluminescence, a larger batch is prepared and recrystallized from an appropriate organic solvent if necessary for evaluation of usefulness for applications.

Triboluminescent Compounds

In another of its aspects, the invention relates to the triboluminescent compounds formed by the method of the present invention. The compounds comprise complexes of lanthanides with 1,3-diketones having bulky substituents on the ends (i.e., bulky R 1 and R2) and one of the following bases (illustrated by R1 , R2, R3, N in equations 1 and 3): tertiary amines, pyridines (including those with fused rings), diamines and amine oxides in ratios of: diketone:base:Ln of 4:4:1 and 3:4:1. The compounds of the present invention are as follows: Triboluminescent compounds having the formula shown in Equation 1 (4:4:1 Ratio) wherein:

R1=C(CH3)3, t-butyl, R2=phenyl, amine=N-ethylpyridine, Ln=Gd;

R1=R2=phenyl, amine=N-ethylpyridine, Ln=Dy;

R1=R2=phenyl, amine=dimethylbenzylamine, Ln=Dy.

Triboluminescent compounds having the formula shown in Equation 3 (3:4:1 Ratio)

wherein

R1=R2=phenyl, amine=N-ethylpyridine, Ln=Gd;

R1=R2=phenyl, amine=dimethylbenzylamine, Ln=Gd;

R1=trifluoromethyl, R2=2-thienyl, amine=N-ethylpyridine, Ln=Gd;

R1=R2=phenyl, amine=N-ethylpyridine, Ln=Dy;

R1=trifluoromethyl, R2=2-thienyl, amine=4-dimethylaminopyridine, Ln=Dy.

### EXAMPLE 2

The following eight previously known triboluminescent compounds were synthesized to demonstrate the efficacy of the method of the present invention:

Triboluminescent compounds having the formula shown in Equation 1 (4:4:1 Ratio)

R=R2=phenyl, R3=R4=R5=ethyl, and Ln=Eu, Gd, or Tb.

R1=phenyl, R3=R4=methyl, R5=phenyl, and Ln=Eu.

Triboluminescent compounds having the formula shown in Equation 3 (3:4:1 Ratio)

R1=R2=t-butyl, amine=dimethylaminopyridine, and Ln=Eu, Dy, Sm, or Tb.

Each of the compounds synthesized by the method of the present invention have applications for sensing or imaging friction, abrasion, wear, strain, elastic or plastic deformation, impulse, impact, or fracture, whether used near, coated on a surface or embedded in a matrix and in devices to image or examine the mechanism of elastic or plastic deformation or fracture. In addition, the photoluminescent property of the compounds renders them useful as phosphors in lamps, television screens and computer monitors.

Having now fully set forth the preferred embodiments and certain modifications of the concept underlying the present invention, various other embodiments as well as certain variations and modifications thereto may obviously occur to those skilled in the art upon becoming familiar with the underlying concept. It is to be understood, therefore, that the invention may be practiced otherwise than as specifically set forth in the appended claims:

I claim:

1. A combinatorial library comprising triboluminescent compounds of the structure: 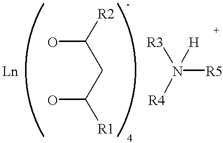
wherein
R1=C(CH3)3, t-butyl, R2=phenyl, amine=N-ethylpyridine, Ln=Gd;
R1=R2=phenyl, amine=N-ethylpyridine, Ln=Dy;
R1=r2=phenyl, amine=dimethylbenzylamine, Ln=Dy.

2. A combinatorial library comprising triboluminescent compounds of the structure: 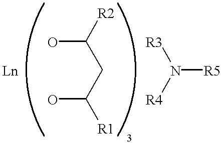
wherein
R1=R2=phenyl, amine=N-ethylpyridine, Ln=Gd;
R1=R2=phenyl, amine=dimethylbenzylamine, Ln=Gd;
R1=trifluoromethyl, R2=2-thienyl, amine=N-ethylpyridine, Ln=Gd;
R1=R2=phenyl, amine=N-ethylpyridine, Ln=Dy;
R1=trifluoromethyl, R2=2-thienyl, amine=4-dimethylaminopyridine, Ln=Dy.

3. A method for synthesizing and screening a combinatorial library comprising the steps of:
combining a diketone reagent, an amine reagent and a lanthanide salt reagent in a reaction vessel;
generating an array of said reaction vessels wherein said reagents differ from one reaction vessel to the next within said array according to a pre-determined variable parameter;
purifying and isolating a reaction product synthesized from a chemical reaction of said reagents in each of said reaction vessels,
testing said reaction products in each of said reaction vessels for evidence of photoluminescence;
mechanically testing said reaction products in each of said reaction vessels for visual evidence of triboluminescence; and
generating an organized grid of distinct reaction products differing according to said variable parameter, said reaction products providing a visual display of the relative triboluminescence of each of said reaction products.

4. The method according to claim 3, wherein said variable parameter comprises varying the identity of said lanthanide salt reagent selected from the group consisting of La, Ce, Pr, Nd, Pm, Sm, Eu, Gd, Tb, Dy, Ho, Er, Tm, Yb, Lu.

5. The method according to claim 3, wherein said variable parameter comprises varying the identity of said amine reagent.

6. The method according to claim 3, wherein said variable parameter comprises varying the identity of said diketone reagent.

7. The method according to claim 3, wherein said variable parameter comprises varying a molar ratio of said diketone, amine and lanthanide salt reagents.

8. The method according to claim 7, wherein said molar ratio of the diketone, amine and lanthanide salt reagents is a 4:4:1 molar ratio.

9. The method according to claim 7, wherein said molar ratio of the diketone, amine and lanthanide salt reagents is a 3:4:1 molar ratio.

10. A tribolumenscent compound of the structure: 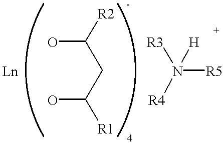
wherein
R1=C(CH3)3, t-butyl, R2=phenyl, amine=N-ethylpyridine, Ln=Gd;
R1=R2=phenyl, amine=N-ethylpyridine, Ln=Dy;
R1=R2=phenyl, amine=dimethylbenzylamine, Ln=Dy.

11. A tribolumenscent compound of the structure: 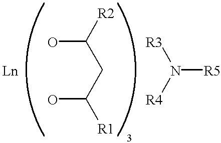
wherein
R1=R2=phenyl, amine=N-ethylpyridine, Ln=Gd;
R1=R2=phenyl, amine=dimethylbenzylamine, Ln=Gd;
R1=trifluoromethyl, R2=2-thienyl, amine=N-ethylpyridine, Ln=Gd;
R1=R2=phenyl, amine=N-ethylpyridine, Ln=Dy;
R1=trifluoromethyl, R2=2-thienyl, amine=4-dimethylaminopyridine, Ln=Dy.
